(19) United States
(12) Patent Application Publication
(10) Pub. No.: US 2024/0382267 A1
(43) Pub. Date: Nov. 21, 2024

(54) METHODS FOR ALIGNING SENSOR-ENABLED PROSTHESIS DURING ROBOTICALLY-ASSISTED ARTHROPLASTY

(71) Applicant: Orthosoft ULC, Montreal (CA)

(72) Inventor: Mara Catalina Aguilera Canon, Calne (GB)

(21) Appl. No.: 18/662,290

(22) Filed: May 13, 2024

Related U.S. Application Data
(60) Provisional application No. 63/467,767, filed on May 19, 2023.

(51) Int. Cl. A61B 34/20; A61B 34/00; A61B 34/30; A61B 90/00; A61B 90/50; A61F 2/46

(52) U.S. Cl. CPC A61B 34/20; A61B 34/25; A61B 34/30; A61F 2/4603; A61B 2090/365; A61B 2090/502; A61F 2002/4633

(57) ABSTRACT

A method for registering output of sensor-enabled implants with a bone axis during robotically-assisted arthroplasty procedures comprises registering anatomy of a patient to a surgical tracking system, determining a bone axis of a bone of the anatomy using the surgical tracking system, preparing the bone to receive a prosthetic implant including an orientation sensor, inserting the prosthetic implant into the bone, obtaining orientation output from the orientation sensor, and shifting the orientation output from the orientation sensor to align with the bone axis. A system for registering output of sensor-enabled implants with a bone axis during robotically-assisted arthroplasty procedures comprises a surgical robot comprising an arm configured to move within a coordinate system, a tracking system configured determine locations of one or more trackers in the coordinate system, a sensor-enabled implant configured to implanted into anatomy and output orientation data, and a controller for the surgical robot.

### CLAIM OF PRIORITY

This application claims the benefit of U.S. Provisional Patent Application Ser. No. 63/467,767, filed on May 19, 2023, the benefit of priority of which is claimed hereby, and which is incorporated by reference herein in its entirety.

### TECHNICAL FIELD

The present disclosure is directed to devices and methods for use in performing a joint arthroplasty, such as knee, hip and shoulder replacement procedures. In examples, the devices and methods can be used to facilitate alignment of sensor-enabled orthopedic implants with anatomy of a patient.

### BACKGROUND

Arthroplasty procedures involve the implantation of medical devices, e.g., orthopedic implants, into anatomy of a patient. Typically, once the medical device is implanted into the patient, or even while it is being implanted, it is difficult to obtain feedback regarding the effectiveness of the implant or the implant procedure. Attempts have been made to obtain data from orthopedic implants using sensors.

Pub. No. US 2018/0125365 to Hunter et al. is titled “Devices, Systems and Methods for Using and Monitoring Medical Devices.”

Pub. No. US 2019/0350518 to Bailey et al. is titled “Implantable Reporting Processor for an Implant.”

### Overview

The present inventor has recognized, among other things, that problems to be solved with sensor-enabled implants involve positioning of the sensor relative to the anatomy. Sensor-enabled implants can include sensors configured to provide motion output relative to an internal frame of reference. For example, sensors included within sensor modules of various prosthetic devices can include one or more 3-axis sensors or accelerometers configured to provide output of movement of the sensor module. It is desirable to implant the sensor module within a known frame of reference such that output of the sensor module can be correlated to kinematic movement of the patient. Typically, the sensor module can be registered to the anatomy of the patient via alignment to the prosthetic device in a known manner. However, due to a variety of factors, the intended orientation of the sensor module can be skewed relative to the anatomy such that data output of the sensor module can be inaccurate or shifted from the desired frame of reference. For example, misalignment of the sensor module to the prosthetic device, misalignment of the prosthetic device to the anatomy, imperfections in resection planes and the like can result in sensor module output being offset from the desired reference frame. The skewed sensor data can be corrected post-operatively. But such post-operative adjustment of the sensor module output can sometimes occur after a period of time before the misalignment is detected, can take multiple recalibration attempts and can be less accurate than if properly aligned from the outset.

The present inventor has recognized that robotic surgical systems can be used to solve problems associated with sensor-enabled implants. In robotic surgical systems, the shape of the anatomy of a patient obtained from patient imaging can be registered with another frame of reference, such as the physical space of an operating room where the robotic surgical system is located, which can be associated with surgical landmarks such as bone landmarks in the anatomy to, for example, help estimate anatomical and kinematic axes. The surgical system can utilize an optical tracking system that can track the location and position of tracking arrays attached to various objects, such as instruments, anatomy and the robotic surgical arm. Robotic surgical arms can be used to hold various instruments in place in a desired orientation relative to both the anatomy and operating room during a procedure so that movement of an instrument in the operating room relative to the anatomy can be tracked on the anatomic imaging based on movement of the robotic surgical arm. As such, robotic surgical systems include a robotic frame of reference in which an anatomic frame of reference of a patient is known. With the present disclosure, the robotic frame of reference of a robotic surgical system can be used to align output of a sensor module to the anatomic frame of reference of the patient. For example, output of a sensor-enabled implant and orientation output from tracking arrays associated with bones and/or kinematic axes can be correlated to register output of the sensor-enabled implant to the anatomic reference frame.

In an example, a method for registering output of a sensor-enabled implant with a bone axis during a robotically-assisted arthroplasty procedure can comprise registering anatomy of a patient to a surgical tracking system, determining a bone axis of a bone of the anatomy using the surgical tracking system, preparing the bone to receive a prosthetic implant including an orientation sensor, inserting the prosthetic implant into the bone, obtaining orientation output from the orientation sensor, and shifting the orientation output from the orientation sensor to align with the bone axis.

In an additional example, a system for registering output of a sensor-enabled implant with a bone axis during a robotically-assisted arthroplasty procedure can comprise a surgical robot comprising an articulating arm configured to move within a coordinate system for the surgical robot, a tracking system configured determine locations of one or more trackers in the coordinate system, a sensor-enabled implant configured to be implanted into anatomy and output orientation data, and a controller for the surgical robot comprising a communication device configured to receive data from and transmit data to the surgical robot, the tracking system and the sensor-enabled implant, a display device for outputting visual information from the surgical robot, the tracking system and the sensor-enabled implant and a non-transitory storage medium having computer-readable instructions stored therein comprising registering anatomy of a patient to a surgical tracking system, determining a bone axis of a bone of the anatomy using the surgical tracking system, obtaining orientation output from an orientation sensor of a sensor-enabled prosthetic implant implanted into bone, and shifting the orientation output from the orientation sensor to align with the bone axis.

### BRIEF DESCRIPTION OF THE DRAWINGS

FIG. 1 is a diagrammatic view of an operating room including a robot-assisted surgical system comprising a robotic arm, a computing system and a tracking system.

FIG. 2 is a schematic view of the robotic arm of FIG. 1 including a resection instrument configured to provide cutting guide functions and serve as a platform for mounting components for additional surgical steps, such as can be used to perform a total knee arthroplasty involving sensor-assisted implants.

FIG. 3A is a perspective view of an example of a sensor-enabled implant comprising a stem for a tibial implant and a sensor module.

FIG. 3B is an exploded view of an electronics assembly that can be used with the sensor module of the sensor-enabled implant of FIG. 3A.

FIG. 4 is a perspective view of the sensor-enabled implant of FIG. 3A attached to a keel of a tibial plate and implanted into an epiphysis region of a tibia.

FIG. 5 is a schematic view of a knee joint comprising a femur and a tibia extending along a femoral axis and a tibial axis disposed relative to a side view of the sensor-enabled implant of FIGS. 3 and 4.

FIG. 6 is a perspective view of the cutting guide of FIG. 2 disposed relative to a tibia to perform a proximal resection.

FIG. 7 is a perspective view of the tibia of FIG. 6 shown relative to a tibial drill guide.

FIG. 8 is a schematic view of a surgical helmet including an augmented reality headset shown interacting with tracking elements attached to anatomy of a patient.

FIG. 9A is a schematic illustration of a display output of the augmented reality headset of FIG. 8.

FIG. 9B is a schematic illustration of a user interface that can be used to adjust the position of a sensor module axis.

FIG. 10 is a block diagram of steps for an exemplary method of aligning and registering a sensor-enabled implant with anatomy.

FIG. 11 is a schematic illustration of a robotic surgical system, a tracking system and a sensor-enabled implant system interacting with each other.

FIG. 12 is a block diagram of an example machine upon which any one or more of the techniques and methods discussed herein may be performed and with which any of the devices discussed herein may be used in accordance with some embodiments.

### DETAILED DESCRIPTION

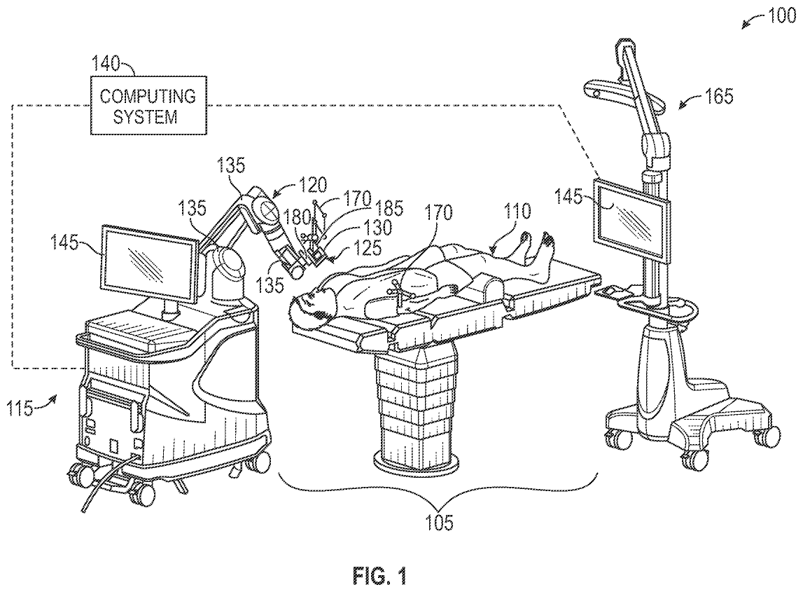

FIG. 1 is a diagrammatic view of surgical system 100 for operation on surgical area 105 of patient 110 in accordance with at least one example of the present disclosure. Surgical area 105 in one example can include a joint and, in another example, can be a bone. Surgical area 105 can include any surgical area of patient 110, including but not limited to the shoulder, knee, hip, head, elbow, thumb, spine, and the like. Surgical system 100 can also include robotic system 115 with one or more robotic arms, such as robotic arm 120. As illustrated, robotic system 115 can utilize only a single robotic arm. Robotic arm 120 can be a 6 degree-of-freedom (DOF) robot arm, such as the ROSA® robot from Medtech, a Zimmer Biomet Holdings, Inc. company. In some examples, robotic arm 120 is cooperatively controlled with surgeon input on the end effector or surgical instrument, such as surgical instrument 125. In other examples, robotic arm 120 can operate autonomously. While not illustrated in FIG. 1, one or more positionable surgical support arms can be incorporated into surgical system 100 to assist in positioning and stabilizing instruments or anatomy during various procedures.

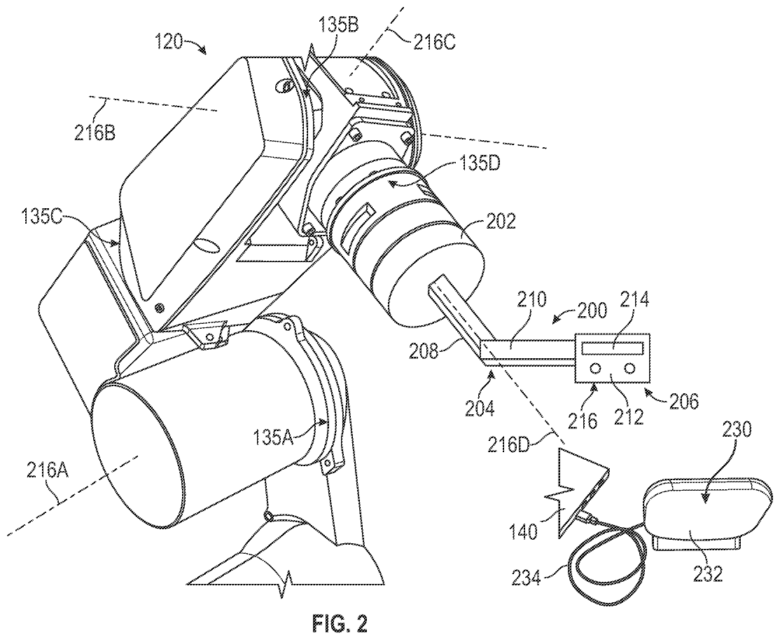

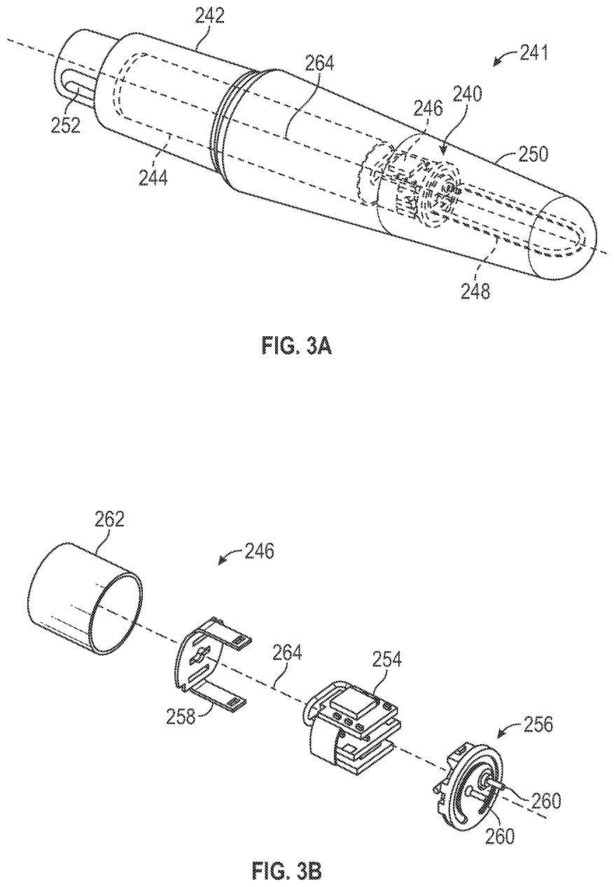

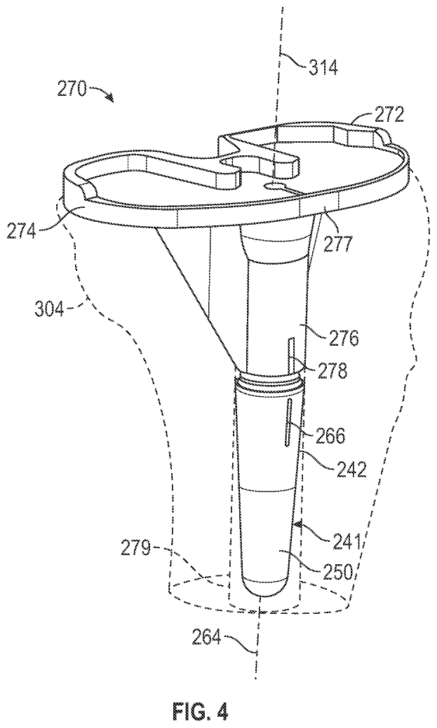

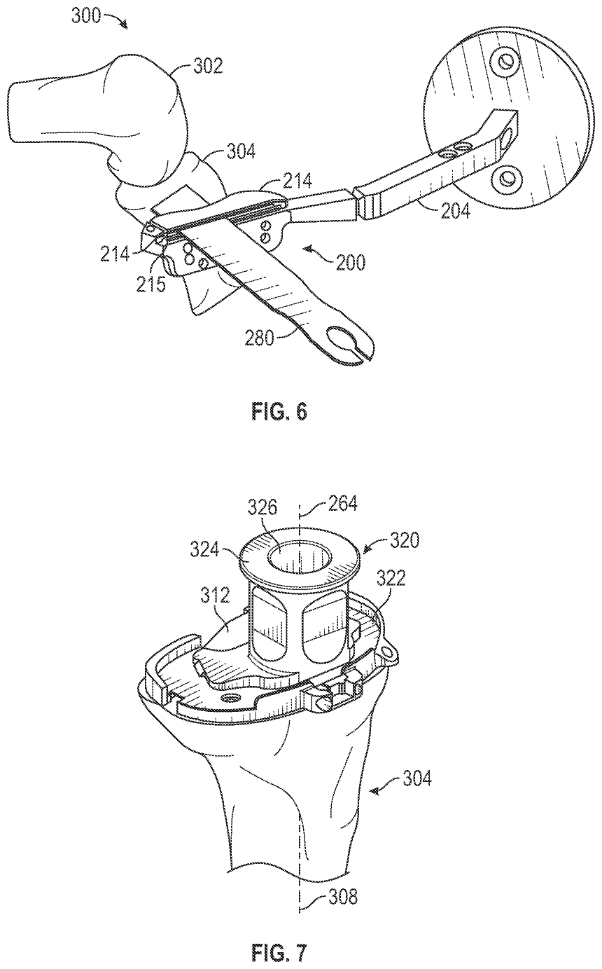

Each robotic arm 120 can rotate axially and radially and can receive an end effector, such as surgical instrument 125, at distal end 130. Surgical instrument 125 can be any surgical instrument adapted for use by the robotic system 115, including, for example, a guide tube, a holder device, a gripping device such as a pincer grip, a burring device, a reaming device, an impactor device such as a humeral head impactor, a pointer, a probe, a cutting guide, an instrument guide, an instrument holder or a universal instrument adapter device as described herein or the like. Surgical instrument 125 can be positionable by robotic arm 120, which can include multiple robotic joints, such as joints 135, that allow surgical instrument 125 to be positioned at any desired location adjacent or within a given surgical area 105. As discussed below, robotic arm 120 can be used with resection guide instrument 200 of FIGS. 2 and 6 to perform a proximal tibial resection for a total knee arthroplasty. Robotic arm 120 can additionally be used with sensor-enabled implants, such as sensor-enabled implant 241 of FIGS. 3 and 4.

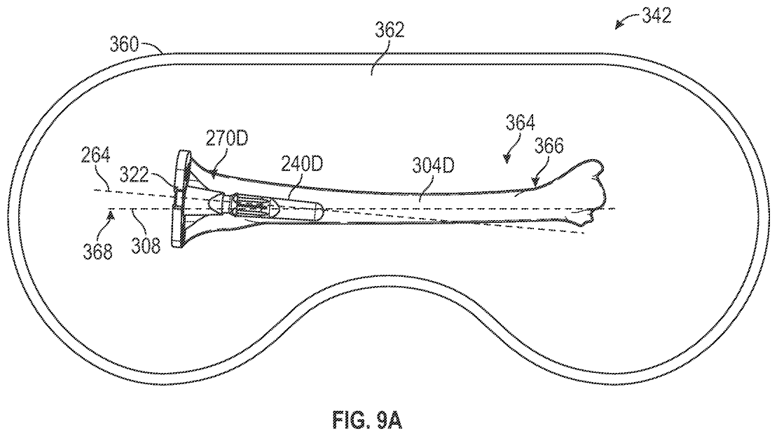

Robotic system 115 can also include computing system 140 that can operate robotic arm 120 and surgical instrument 125. Computing system 140 can include at least memory, a processing unit, and user input devices, as will be described herein. Computing system 140 and tracking system 165 can also include human interface devices 145 for providing images for a surgeon to be used during surgery. Computing system 140 is illustrated as a separate standalone system, but in some examples computing system 140 can be integrated into robotic system 115. Human interface devices 145 can provide images, including but not limited to three-dimensional images of bones, glenoids, knees, joints, and the like. In examples, human interface device 145 can be used to display an axis of a sensor module for a sensor-enabled implant, such as sensor module axis 264 of FIG. 3A. Human interface devices 145 can include associated input mechanisms, such as a touch screen, foot pedals, or other input devices compatible with a surgical environment. As discussed below, computing system 140 can interact with base station 230 of FIG. 2 and surgical helmet 340 of FIG. 8.

Computing system 140 can receive pre-operative, intra-operative and post-operative medical images. These images can be received in any manner and the images can include, but are not limited to, computed tomography (CT) scans, magnetic resonance imaging (MRI), two-dimensional x-rays, three-dimensional x-rays, ultrasound, and the like. These images in one example can be sent via a server as files attached to an email. In another example the images can be stored on an external memory device such as a memory stick and coupled to a USB port of the robotic system to be uploaded into the processing unit. In yet other examples, the images can be accessed over a network by computing system 140 from a remote storage device or service.

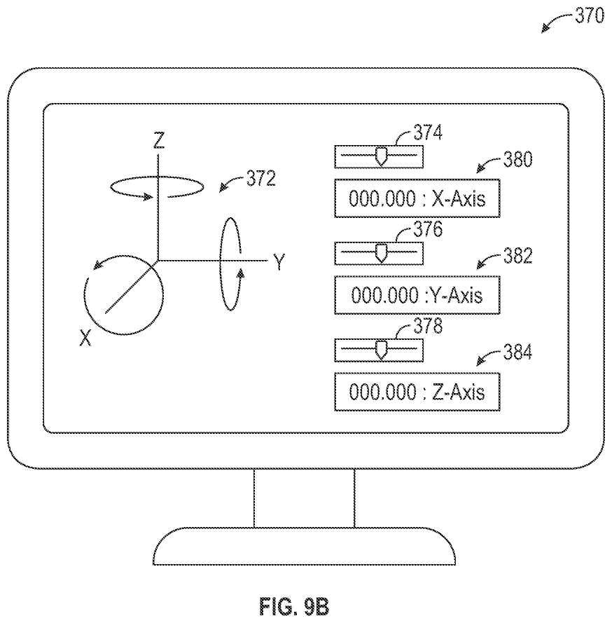

After receiving one or more images, computing system 140 can generate one or more virtual models related to surgical area 105. Alternatively, computing system 140 can receive virtual models of the anatomy of the patient prepared remotely. Specifically, a virtual model of the anatomy of patient 110 can be created by defining anatomical points within the image(s) and/or by fitting a statistical anatomical model to the image data. The virtual model, along with virtual representations of implants, can be used for calculations related to the desired location, height, depth, inclination angle, or version angle of an implant, stem, acetabular cup, glenoid cup, total ankle prosthetic, total and partial knee prosthetics, surgical instrument, or the like to be utilized in surgical area 105. As discussed below, digital model 304D of tibia 304 is shown in FIG. 9A. In another procedure type, the virtual model can be utilized to determine resection locations on femur and tibia bones for a partial knee arthroplasty. In a specific example, the virtual model can be used to determine the orientation of a sensor-enabled implant relative to anatomic landmarks. The virtual model can also be used to determine bone dimensions, implant dimensions, bone fragment dimensions, bone fragment arrangements, and the like. Any model generated, including three-dimensional models, can be displayed on human interface devices 145 for reference during a surgery or used by robotic system 115 to determine motions, actions, and operations of robotic arm 120 or surgical instrument 125. Known techniques for creating virtual bone models can be utilized, such as those discussed in U.S. Pat. No. 9,675,461, titled “Deformable articulating templates” or U.S. Pat. No. 8,884,618, titled “Method of generating a patient-specific bone shell” both by Mohamed Rashwan Mahfouz, as well as other techniques known in the art.

Computing system 140 can also communicate with tracking system 165 that can be operated by computing system 140 as a stand-alone unit. Surgical system 100 can utilize the Polaris optical tracking system from Northern Digital, Inc. of Waterloo, Ontario, Canada. Additionally, tracking system 165 can comprise the tracking system shown and described in Pub. No. US 2017/0312035, titled “Surgical System Having Assisted Navigation” to Brian M. May, which is hereby incorporated by this reference in its entirety. Tracking system 165 can monitor a plurality of tracking elements, such as tracking elements 170, affixed to objects of interest to track locations of multiple objects within the surgical field using a tracker, such as a camera. Tracking system 165 can interact with tracking element 348 and tracking element 350 of FIG. 8 to determine the relative positions and orientations of tibia 304 and femur 302. Tracking system 165 can function to create a virtual three-dimensional coordinate system within the surgical field for tracking patient anatomy, surgical instruments, or portions of robotic system 115. Tracking elements 170 can be tracking frames including multiple IR reflective tracking spheres, or similar optically tracked marker devices. In one example, tracking elements 170 can be placed on or adjacent one or more bones of patient 110. In other examples, tracking elements 170 can be placed on robotic arm 120, surgical instrument 125, and/or an implant to accurately track positions within the virtual coordinate system associated with surgical system 100. In each instance tracking elements 170 can provide position data, such as patient position, bone position, joint position, robotic arm position, implant position, or the like.

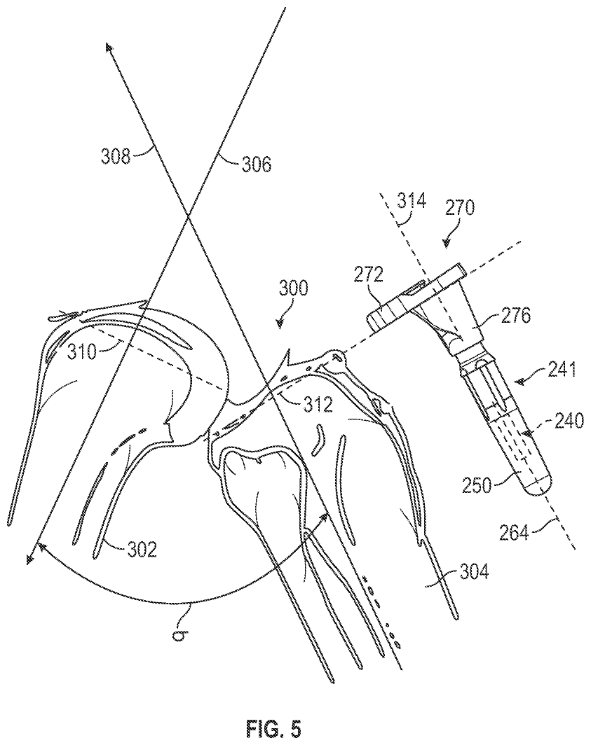

Robotic system 115 can include various additional sensors and guide devices. For example, robotic system 115 can include one or more force sensors, such as force sensor 180. Force sensor 180 can provide additional force data or information to computing system 140 of robotic system 115. Force sensor 180 can be used by a surgeon to cooperatively move robotic arm 120. For example, force sensor 180 can be used to monitor impact or implantation forces during certain operations, such as insertion of an implant stem into a humeral canal. Monitoring forces can assist in preventing negative outcomes through force fitting components. In other examples, force sensor 180 can provide information on soft-tissue tension in the tissues surrounding a target joint. In examples, robotic system 115 can also include laser pointer 185 that can generate a laser beam that is used for alignment of implants during surgical procedures. In examples, laser pointer 185 can be used to generate images of sensor module axis 264 (FIG. 3A) and tibial axis 308 (FIG. 5) onto a tibia of patient 110.

In order to ensure that computing system 140 is moving robotic arm 120 in a known and fixed relationship to surgical area 105 and patient 110, the space of surgical area 105 and patient 110 can be registered to computing system 140 via a registration process involving registering fiducial markers attached to patient 110 with corresponding images of the markers in patient 110 recorded preoperatively or just prior to a surgical procedure. For example, a plurality of fiducial markers can be attached to patient 110, images of patient 110 with the fiducial markers can be taken or obtained and stored within a memory device of computing system 140. Subsequently, patient 110 with the fiducial markers can be moved into, if not already there because of the imaging, surgical area 105 and robotic arm 120 can touch each of the fiducial markers. Engagement of each of the fiducial markers can be cross-referenced with, or registered to, the location of the same fiducial marker in the images. In additional examples, patient 110 and medical images of the patient can be registered in real space using contactless methods, such as by using a laser rangefinder held by robotic arm 120 and a surface matching algorithm that can match the surface of the patient from scanning of the laser rangefinder and the surface of the patient in the medical images. As such, the real-world, three-dimensional geometry of the anatomy attached to the fiducial markers can be correlated to the anatomy in the images and movements of surgical instrument 125 attached to robotic arm 120 based on the images will correspondingly occur in surgical area 105.

Subsequently, other instruments and devices attached to surgical system 100 can be positioned by robotic arm 120 into a known and desired orientation relative to the anatomy. For example, robotic arm 120 can be coupled to resection guide instrument 200 of FIG. 2, that can be used to guide resections on multiple bones (e.g., proximal tibia and distal femur) and that allows other instruments (e.g., a finishing guide or posterior cut guide) to be attached to robotic arm without having to individually couple each instrument to robotic arm in succession and without the need for individually registering each attached instrument with the coordinate system. Robotic arm 120 can move resection guide instrument 200 relative to anatomy of the patient such that the surgeon can, after adding and removing another instrument to the guide instrument as needed, perform the desired interaction with the patient at specific locations called for by the surgical plan with the attached instrument.

In the present application, surgical system 100 can be configured to operate with sensor-enabled implants such that the three-dimensional space of surgical area 105 can be correlated to orientation and motion data of the sensor-enabled implant to allow a surgeon to ensure alignment of the data of the sensor-enabled implant with the anatomy of the patient.

FIG. 2 is a schematic view of robotic arm 120 of FIG. 1 including resection guide instrument 200, which can be positioned by robotic arm 120 relative to surgical area 105 (FIG. 1) in a desired orientation according to a surgical plan, such as a plan based on preoperative imaging or based, at least partially, on intra-operative planning. Resection guide instrument 200 can comprise tool base 202, extension arm 204 and guide block 206. Extension arm 204 can comprise first segment 208 and second segment 210, as well as additional segments in other examples. Guide block 206 can comprise body 212, guide surface 214 and interface 216. In an example, guide block 206 can be configured as a resection block for use in a partial knee arthroplasty and, as such, guide block 206 can be used to perform a proximal resection of a tibial plateau, as shown in FIG. 6, and a distal resection of a femoral condyle.

Robotic arm 120 can include joint 135A that permits rotation about axis 216A, joint 135B that can permit rotation about axis 216B, joint 135C that can permit rotation about axis 216C and joint 135D that can permit rotation about axis 216D.

In order to position resection guide instrument 200 relative to anatomy of patient 110 (FIG. 1), surgical system 100 (FIG. 1) can manipulate robotic arm 120 automatically by computing system 140 or a surgeon manually operating computing system 140 to move resection guide instrument 200 to the desired location, e.g., a location called for by a surgical plan to align an instrument relative to the anatomy. For example, robotic arm 120 can be manipulated along axis 216A to axis 216D to position resection guide instrument 200 such that guide block 206 is located in a desired location relative to the anatomy. As such, a step of a surgical procedure can be performed, such as by using guide surface 214. However, subsequent steps of the surgical procedure can be performed with resection guide instrument 200 without having to uncouple resection guide instrument 200 from robotic arm 120. For example, other instruments can be attached to guide block 206 at interface 216. Other instruments attached at interface 216 can be used without having to re-register an additional instrument to the coordinate system because the dimensions and geometries of resection guide instrument 200 and other instruments to be used therewith can be known by surgical system 100 (FIG. 1) such that the locations of guide block 206 and instruments attached thereto can be calculated by surgical system 100 as robotic arm 120 moves throughout the coordinate system.

Robotic arm 120 can be separately registered to the coordinate system of surgical system 100, such via use of a tracking element 170 (FIG. 1). Fiducial markers can additionally be separately registered to the coordinate system of surgical system 100 via engagement with a probe having a tracking element 170 attached thereto. Resection guide instrument 200 can be registered to the coordinate system via coupling with robotic arm. Furthermore, output of sensor-enabled implant 241 (FIGS. 3A and 4) can be registered to the coordinate system of surgical system 100 via base station 230. Base station 230 can comprise a wireless communication device 232 for receiving output of sensor module 240. Base station 230 can comprise cable or wire 234 for connecting to computing system 140 of surgical system 100. As such, some or all of the components of surgical system 100 can be individually registered to the coordinate system (with or without the aid of tracking elements) and, if desired, movement of such components can be continuously or intermittently tracked with a tracking element 170.

In some robotic procedures, instruments can be separately and individually tracked using an optical navigation system that, under ideal conditions, alleviate the need for precisely maintaining axis 216D and the location of an instrument along axis 216D through a surgical procedure or surgical task, as the optical navigation system can provide the surgical computer system information to compensate for any changes. However, as optical navigation systems require line-of-sight with the instruments to be maintained, there is a significant advantage in not requiring instruments to be navigated (or at least not constantly navigated). Resection guide instrument 200 allows multiple instruments to be registered to robotic system 115 without the need for individually tracking each instrument. Robotic system 115 can know the precise location of robotic arm 120, and the geometry and dimensions of resection guide instrument 200 can be registered to robotic system 115. As such, the location of guide block 206 in the surgical space can be determined as robotic arm 120 moves guide block 206 within the surgical space. Furthermore, robotic system 115 can be provided with, such as within a non-transient computer-readable storage medium, the geometry and dimensions of instruments configured to be attached to guide block 206 such that the locations of attachment instruments can also be tracked as robotic arm 120 moves. Thus, individual tracking or registration of the attachment instruments can be avoided if desired. Additionally, robotic system 115 can be provided with, such as within a non-transient computer-readable storage medium, the geometry and dimensions of sensor-enabled implant 241 (FIG. 4) and tibial component 270 (FIG. 5) such that the geometry and location of tibial component 270 and sensor-enabled implant 241 including sensor module 240 can be registered to the anatomy when implanted.

FIG. 3A is a perspective view of sensor module 240 for sensor-enabled implant 241. Sensor module 240 can comprise outer casing 242, battery 244, electronics assembly 246, and antenna 248. Outer casing 242 can comprise radome 250 that can be used to cover and protect antenna 248 to allow sensor module 240 to receive and transmit information via a wireless signal. Outer casing 242 can include set-screw engagement hole 252, which can be utilized to physically attach sensor module 240 to tibial plate 272 (FIG. 4). In the example of FIG. 3A, sensor-enabled implant 241 comprises a tibial stem such that outer casing 242 comprises an elongate cylinder-like body that can be inserted into a tibia when attached to tibial plate 272 (FIG. 4).

FIG. 3B is an exploded view of electronics assembly 246 that can be used with sensor module 240 of FIG. 3A. Electronics assembly 246 includes a printed circuit assembly (PCA), such as PCA 254, which can be physically attached and electrically connected to header assembly 256. For the illustrated example, PCA 254 includes three rigid printed circuit boards (PCBs) with electronic components (e.g., integrated circuit chips) mounted thereon and electrically interconnected utilizing flexible conductive wiring, such as, for example, flexible flat cable fabricated as an inner layer of the PCB (e.g., rigid-flex). The three rigid printed circuit boards of PCA 254, which can be folded over so as to overlap each other and thus save physical space, can be characterized as a tri-fold printed circuit assembly. Electronics assembly 246 can also include printed circuit assembly clip 258, which can be utilized to physically affix PCA 254 to one side of the header assembly 256. Printed circuit assembly clip 258 can be made of a suitable sturdy and corrosion-resistant material, such as, for example, titanium (Ti) and the like. The side of header assembly 256 opposite PCA 254 can include two antenna connections 260, which can be utilized as mounting points and electrical connections for an antenna. Thus, header assembly 256 can function to electrically and physically connect an antenna to, for example, a radio transmitter circuit mounted on one of the printed circuit boards of the PCA 254. Electronics assembly 246 can also include case 262, which can be physically affixed to header assembly 256 and thereby utilized to enclose and hermetically seal PCA 254 and printed circuit assembly clip 258 within. For example, case 262 can be made of a suitable sturdy and corrosion-resistant material, such as titanium (Ti) and the like. Outer casing 242 and radome 250 can be attached in an end-to-end configuration such that both components extend along sensor module axis 264.

Electronics assembly 246 can include, circuits, pressure sensors, temperature sensors, pedometers, on-board volatile memory (e.g., random-access memory (RAM), dynamic RAM (DRAM), or static RAM (SRAM)) and nonvolatile memory (e.g., read-only memory (ROM), programmable ROM (PROM, electrically programmable ROM (EPROM), and electrically erasable and programmable ROM (EEPROM)). Electronics assembly 246 can include switches to couple these components. Electronics assembly 246 can comprise a microcontroller, a microprocessor, or any other computing circuit, such as a Silicon Labs® EFM32HG microcontroller IC.

Electronics assembly 246 can include an inertial measurement circuit that includes one or more sensors for acquiring data related to the motion of sensor module 240 and a prosthesis attached thereto, such as tibial component 270 (FIG. 4). In examples, the inertial measurement circuit can include one or more accelerometers, gyroscopes, pedometers, and magnetometers that are respectively configured to sense and measure linear and rotational accelerations, step counts, and magnetic fields that the prosthesis experiences or to which the prosthesis is exposed. In examples, the inertial measurement circuit can include three accelerometers and three gyroscopes, one accelerometer and gyroscope for each dimension of linear (X, Y, Z) and rotational (rotation about X axis, rotation about Y axis, rotation about Z axis) freedom, respectively, that the implanted prosthesis possesses, or is configured to possess. By analyzing the information generated by these sensors while a patient, or other subject, in which the prosthesis is implanted, is moving, one can determine whether the prosthesis is functioning properly, and can predict when the prosthesis should be replaced.

In examples, sensor module 240 can be constructed according to the teachings of US 20190350518 A1 to Bailey et al., titled “Implantable Reporting Processor for an Alert Implant,” the contents of which are hereby incorporated into the present application in their entirety.

Output of sensor module 240 is thus correlated to an internal X, Y, Z frame of reference or a sensor frame of reference. In examples, one of the axes can extend along sensor module axis 264, with the other two axes extending in a plane orthogonal thereto. As shown in FIG. 4, outer casing 242 can be provided with hash mark 278 to provide an indication of the direction of one of the axes relative to sensor module axis 264. As such, the direction of each of the X, Y and Z axes can be physically determined from the exterior of sensor-enabled implant 241.

FIG. 4 is a perspective view of tibial component 270 that can be utilized with sensor-enabled implant 241 having sensor module 240 of FIGS. 3A and 3B. The present application is described with reference to tibial component 270, but can be utilized with other prosthetic implants, such as shoulder implants (including humeral and scapular implants), hip implants (including pelvic and femoral implants), knee implants (including femoral and tibial implants), as well as others. The illustrated example of sensor-enabled implant 241 works particularly well with implants having stem configured for insertion into long bones, such as tibial implants for knee prosthetics, femoral implants for knee prosthetic and humeral implants for shoulder prosthetics. However, sensor-enabled implant 241 can have other form factors for use in other prosthetic constructs. Tibial component 270 can comprise tibial plate 272, keel 274 and tibial extension 276. Tibial component 270 can be attached to tibia 304, which can be reamed to form bone socket 279.

Tibial plate 272 can be a base plate section of an artificial knee joint (prosthesis) that can be implanted during a surgical procedure, such as a total knee arthroplasty (TKA). Prior to, or during the surgical procedure, sensor-enabled implant 241 can be physically attached to tibial plate 272 via coupling to tibial extension 276 via suitable means, such as threaded engagement, snap fit and the like. Outer casing 242 can include set-screw engagement hole 252, which can be utilized to physically attach sensor-enabled implant 241 to tibial plate 272. It is understood that the mechanism for affixing sensor module 240 to tibial component 270 or other implant can also include threaded fasteners as well as a variety of clips and locking mechanisms. In examples, sensor-enabled implant 241 can be attached to tibial component 270 such that sensor module axis 264 aligns with stem axis 314. A surgical plan for patient 110 (FIG. 1) can be prepared to implant tibial component 270 into tibia 304 such that stem axis 314 aligns with the anatomic axis of tibia 304.

In order to facilitate registration of the output of sensor module 240 with tibial component 270, alignment marks or indicia can be included on each component. For example, outer casing 242 can include hash mark 266 and tibial extension 276 can include hash mark 278. Hash mark 266 and hash mark 278 can extend parallel to sensor module axis 264. Hash mark 266 and hash mark 278 can comprise markings, such as ink or paint added to the exterior of outer casing 242 and tibial extension 276. In additional examples, hash mark 266 and hash mark 278 can comprise etchings or depressions extending into outer casing 242 and tibial extension 276 or build-ups or protrusions extending from outer casing 242 and tibial extension 276. Hash mark 266 and hash mark 278 can be oriented relative to each other such that output of sensor module 240 is referenced to an orientation of tibial component 270. In examples, hash mark 278 can be located on the anterior-most portion of tibial extension 276. Thus, hash mark 278 can be aligned with hash mark 266 to ensure that the anterior-posterior axis of tibial component 270 is aligned with the axis of sensor module extending in the direction of hash mark 266 as described with reference to FIGS. 3A and 3B. As such, in examples, x-axis output of sensor module 240 can be configured to align with hash mark 266 to provide forward (anterior-posterior) movements of tibial component 270 and y-axis and z-axis outputs can be appropriately oriented in orthogonal directions to provide upward (superior-inferior) movements and sideways (medial-lateral) movements, when tibial component 270 is implanted into tibia 304. Tibial plate 272 can include anterior surface or indicator 277 that can align with hash mark 278 at the anterior-most portions of tibial component 270. As such, the orientation of tibial component 270 relative to tibia 304 can be visualized by a surgeon during implantation. Indicator 277 can provide visual feedback as to the location of hash mark 278 when tibial extension 276 is obscured or concealed within tibia 304.

FIG. 5 is a schematic view of knee joint 300 comprising femur 302 and tibia 304 extending along femoral axis 306 and tibial axis 308, respectively. Femur 302 and tibia 304 can comprise anatomy of patient 110 (FIG. 1). Knee joint 300 is disposed relative to tibial component 270 and sensor module 240 of FIGS. 3A-4. In order to perform a total knee arthroplasty, a distal resection is performed on femur 302 to produce distal resection plane 310 and a proximal resection is performed on tibia 304 to produce proximal resection plane 312. Femoral axis 306 can be disposed at an angle to distal resection plane 310 and tibial axis 308 can be disposed at an angle to proximal resection plane 312. Femoral axis 306 and tibial axis 308 can be disposed at angle σ relative to each other. Tibial component 270 is implanted in tibia 304 such that tibial plate 272 mates flush with proximal resection plane 312. Tibial extension 276 extends distally from tibial plate 272 along stem axis 314. Stem axis 314 can extend non-parallel to the bottom or inferior surface of tibial plate 272. When tibial component 270 is implanted in tibia 304, stem axis 314 can be offset and angled relative to tibial axis 308.

As discussed above, sensor module 240 can be configured to output three-dimensional orientation information in an X, Y, Z coordinate system relative to sensor module axis 264. Output of sensor module 240 can be registered to the orientation of tibial component 270, such as via the alignment of hash mark 266 with hash mark 278, as shown in FIG. 4. Tibial component 270 can be registered to the anatomy of tibia 304 via flush engagement of tibial plate 272 with proximal resection plane 312, as well as by positioning of indicator 277 relative to anatomic landmarks on tibia 304, such as the tibial tuberosity or soleal line.

It can be desirable for tibial component 270 and sensor module 240 to align with tibia 304 in a known orientation so that output of sensor module 240 is properly registered with the kinematic reference from of knee joint 300 defined by tibial axis 308 and femoral axis 306. Proper positioning of tibial component 270 can be desirable to allow for natural kinematic interaction between tibia 304 and femur 302. Understanding of the kinematic interaction between tibia 304 and femur 302 can be obtained by output of sensor module 240. As such, it is important for the output of sensor module 240 to be suitably referenced relative to tibial axis 308 so that the kinematic analysis of knee joint 300 is properly understood. As mentioned above, the alignment of sensor module 240 with tibial component 270 and the alignment of tibial component 270 can be interfered with or altered by various factors including misalignment between hash mark 266 and hash mark 278, misalignment of tibial plate 272 with proximal resection plane 312, the imperfection on or the location of proximal resection plane 312 and others. With the present disclosure, sensor output of sensor module 240 can be communicated to surgical system 100 (FIG. 1) so that output of sensor module 240 can be registered to the frame of reference of surgical system 100, which is registered to the anatomy of patient 110 (FIG. 1). Output of sensor module 240 can thereafter be registered to tibial axis 308 physically or digitally. Physical registration of sensor module 240 to tibial axis 308 can involve physically moving sensor module 240 via movement of tibial component 270 to obtain the desired output orientation of sensor module 240. Digital registration of sensor module 240 can involve digitally shifting the output of sensor module 240, such as by applying a registration factor (e.g., a numerical correction along the X, Y and Z axes), to obtain the desired output orientation of sensor module 240. The numerical correction can be automatically determined by computing system 140, such as by moving tibia 304 through a series of movements to provide computing system 140 of surgical system with a set of reference data points, or manually by a surgeon by movement of a virtual representation of sensor module axis 264 with virtual representation of tibial axis 308, such as by using human interface device 145 (FIG. 1) or surgical helmet 340 (FIG. 9A). The desired output orientation of sensor module 240 can be when sensor module axis 264 aligns with tibial axis 308, as discussed herein.

FIG. 6 illustrates resection guide instrument 200 that can be used to perform a proximal tibial resection in accordance with some examples of the present disclosure. In examples, resection guide instrument 200 can include guide surface 214 to perform a first cut and guide surface 215 to perform a second cut. Resection guide instrument 200 can be affixed to a distal end of robotic arm 120 (FIG. 2). In examples, cutting device 280 can perform the tibial resection as shown in FIG. 6. Cutting device 280 can comprise a reciprocating and oscillating blade having cutting teeth at the distal end thereof. Robotic arm 120 can position resection guide instrument 200 into a specific orientation relative to tibia 304 as discussed herein. Cutting device 280 can be used to produce proximal resection plane 312 of FIG. 5. In examples, resection guide instrument 200 can positioned so that proximal resection plane 312 is orthogonal to tibial axis 308 (FIG. 5). However, as mentioned, it can be possible for proximal resection plane 312 to be slightly skewed from being orthogonal to tibial axis 308 due to imperfections in the planning process, imperfections in the anatomy, imperfections in executing the surgical plan and the like. Nonetheless, the surgical procedure can proceed to the next step of preparing tibia 304 for tibial component 270.

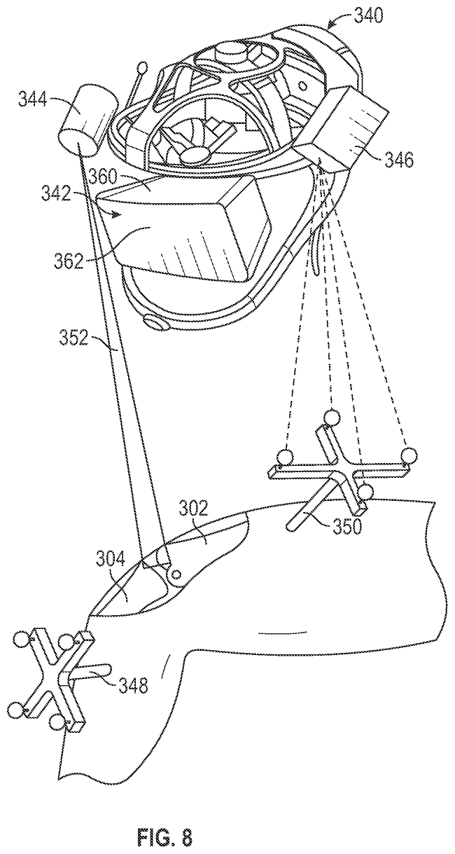

FIG. 7 is a perspective view of tibia 304 of FIG. 6 shown relative to tibial drill guide 320. Tibial drill guide 320 can comprise trial plate 322 and guide sleeve 324 having guide aperture 326. Tibial drill guide 320 can be used to form a passage within tibia 304 into which sensor module 240 and tibial extension 276 can be inserted. Tibial drill guide 320 can be used to produce bone socket 279 of FIG. 4. Tibial drill guide 320 can be shaped to allow tibial component 270 to assemble with tibia 304 in a known orientation such that stem axis 314 will be disposed relative to tibial axis 308 in a known relationship, which helps correlate the output of sensor module 240 with anatomic movements of tibia 304. For example, the bottom of trial plate 322 can be planar to mate flush with proximal resection plane 312. Likewise, the anterior-most point of trial plate 322 can be positioned at the anterior-most point of tibia 304. However, as mentioned, it can be possible for tibial drill guide 320 to be slightly skewed from being aligned with proximal resection plane 312 due to imperfections in the planning process, imperfections in the anatomy, imperfections in executing the surgical plan and the like. Nonetheless, the surgical procedure can proceed to the next step of assembling tibial component 270 and sensor module 240 with tibia 304.

FIG. 8 is a schematic view of surgical helmet 340 including augmented reality headset 342. Surgical helmet 340 can comprise projector 344 (a helmet-mounted projector) and optical locator 346 (a helmet-mounted optical locator). Surgical helmet 340 can be configured to interact with surgical system 100 (FIG. 1).

In examples, projector 344 can comprise a so-called pico projector, or pocket projector, which may be any hand-held sized, commercially available projector capable of emitting a light source, such as a laser or LED light. Optical locator 346 can operate to determine the three-dimensional position of tracking element 348 and tracking element 350 within surgical area 105 (FIG. 1) as has been described herein. Optical locator 346 can additionally be used to track movement of hands of the wearer of surgical helmet 340. In examples, the hands can be tracked with or without the use of optical tracking elements, similar to tracking element 348 and tracking element 350, but miniaturized for incorporation on gloves or the like. As such, hands of a user can be moved to provide input to surgical system to, for example, adjust the position of sensor module axis 264, as well as to achieve other interactions with surgical system 100.

Positioning or locating optical locator 346 directly on surgical helmet 340 can ensure that arrays or tracking elements within the field of view of a surgeon wearing surgical helmet 340 will always be recognized by the navigation system, thus allowing the navigation system to be looking up information relevant to those arrays, and the instruments and tools connected to those arrays, in memory of computing system 140. Positioning or locating projector 344 directly on surgical helmet 340 can ensure that the instructions generated by beam 352 will always remain in the field of view of the surgeon and that the orientation of the instructions will be correlated to the point of view of the surgeon, e.g., any letters or text produced by beam 352 will not be upside down.

Projector 344 can use beam 352 to project various instructions from the surgical plan based on, for example, the instrument that surgeon is holding in his or her hand, such as cutting device 280 (FIG. 6) and tibial drill guide 320 (FIG. 7). The instructions depicted by beam 352 can include various landmarks, alignment axis, and resection planes for projection onto femur 302 and tibia 304 of patient 110 (FIG. 1) to provide visual instructions to a surgeon based on the surgical plan using information stored in the navigation system for each tool or instrument based on the location of each tool or instrument determined by an optical locator and the appropriately correlated array for that particular tool or instrument. Projector 344 can operate to project an indication of tibial axis 308 for tibia 304 (FIG. 5) and an indication of sensor module axis 264 of sensor module 240 (FIG. 5) onto tibia 304 to provide visual instructions to a surgeon as to the orientation of the output of sensor module 240 relative to tibia 304, similar to what is shown for augmented reality headset 342 in FIG. 9. Additionally, augmented reality headset 342 can include goggles 360 having heads-up display screen 362 to provide virtual indicia on tibia 304, as shown in FIG. 9A, that can be used to provide similar indicia for the surgeon as just described.

FIG. 9A is a schematic illustration of a display output 364 of augmented reality headset 342 of FIG. 8. Goggles 360 can comprise a frame for supporting heads-up display screen 362. Goggles 360 can be supported by a frame of surgical helmet 340 or can be directly supported by a head of a user, e.g., a surgeon, via appropriate earpieces or head straps. Heads-up display screen 362 can comprise a lens, e.g., glass or polycarbonate, upon which an image can be projected. One or more cameras attached to goggles 360 or surgical helmet 340 can be used to view the surrounding environment, e.g., the reality, in the field of view of surgical helmet 340. The surrounding environment can be projected onto heads-up display screen 362. In examples, the camera comprising optical locator 346 can be used to record the environment for providing a video transmission to heads-up display screen 362. Additionally, goggles 360 or surgical helmet 340 can include one or more projectors that can project output, e.g., the augmentation, onto heads-up display screen 362. In examples, projector 344 can be used to project the augmented reality output or indicia. In examples, projectors or other components can be included within goggles 360 to generate augmented reality output, e.g., virtual axes, onto display screen. As such, the augmented reality of the one or more projectors can be displayed on top of the video output of the one or more cameras. In additional examples, heads-up display screen 362 can allow for a user, e.g., a surgeon to see the surrounding environment, e.g., the reality, through heads-up display screen 362 without the use of projected video images. However, in such configurations, heads-up display screen 362 can having coatings or other features to allow projected or electronic indicia to be visible on heads-up display screen 362 to provide the augmented reality output.

Display output 364 can comprise video display 366 and augmented reality display 368. Video display 366 can comprise video representations of tibia 304, sensor module 240 and tibial component 270, shown as digital tibia 304D, digital sensor module 240D and digital tibial component 270D, respectively. However, as discussed above, heads-up display screen 362 can be configured to allow a wearer to see-through heads-up display screen 362 to view tibia 304, sensor module 240 and tibial component 270 directly. Augmented reality display 368 can comprise digital representations of tibial axis 308 for tibia 304 (FIG. 5) and sensor module axis 264 of sensor module 240 (FIG. 5). Additionally, digital representations of tibial component 270 and sensor module 240 can be displayed to facilitate visualization since tibial component 270 and sensor module 240 can be obscured by tissue.

As discussed herein, a surgeon can 1) physically manipulate sensor module 240 relative to tibial component 270 to change the orientation of sensor module axis 264 (such as by removing tibial component 270 from tibia 304 and adjusting the position or coupling of sensor module 240), 2) physically manipulate tibial component 270 relative to tibia 304, and 3) digitally moving sensor module axis 264 using computing system 140 (FIG. 1). Options 1) and 2) can be performed to ensure proper assembly of sensor module 240 with tibial component 270 and proper insertion of tibial component 270 according to the surgical plan, respectively. However, the assembly of sensor module 240 and tibial component 270 may be acceptable and it may not be possible to alter the position of tibial component 270 due to the surgical plan. Thus, option 3) can be performed to digitally offset or calibrate the output of sensor module 240 to match the desired reference frame, e.g., tibial axis 308. Option 3) can be performed by a user or surgeon utilizing a human interface device to manipulate the position of sensor module axis 264 displayed as described herein, such as on a display screen, using projected illumination light, or virtually using heads-up display screen 362. For example, a user can do one or both of pivoting or rotating a representation of sensor module axis 264 on human interface device 145 (FIG. 1) or by using hand gestures in conjunction with surgical helmet 340, and inputting values for X, Y and Z axis numerical offsets into human interface device 145. As a user manipulates the representation of sensor module 308 on a display screen or enters different values for the X, Y and Z axis numerical offsets the position of the representation of sensor module axis 264 can change. A user can continue to digitally manipulate the representation of sensor module axis 264 until sensor module axis 264 aligns with tibial axis 308 or another landmark or reference potin. For example, rather than aligning sensor module axis 264 with tibial axis 308, sensor module axis 264 can be aligned with anatomic features on tibia 304, such as the tibial tuberosity or soleal line. In additional examples, computing system 140 can automatically align sensor module axis 264 with tibial axis 308. Once sensor module axis 264 is positioned in the desired orientation, a correction factor can be supplied to sensor module 240, such as by computing system 140 sending a wireless signal to sensor module 240 through base station 230 (FIG. 2). The correction factor can be stored in memory of electronics assembly 246 (FIG. 3B) so that output of sensor module 240 can be customized for a specific patient.

FIG. 9B is a schematic illustration of user interface 370 that can be used to adjust the position of a sensor module axis 264. In examples, user interface 370 can comprise one of human interface devices 145 (FIG. 1). In examples, user interface 370 can comprise a touch-screen display. User interface 370 can display coordinate system icon 372 having an x-axis, a y-axis and a z-axis. User interface 370 can display numerical values for the orientation of each axis, such as x-axis values 380, y-axis values 382 and z-axis values 384. User interface 370 can display user inputs to allow for the adjustment of the x-axis values, the y-axis values and the z-axis values. In examples, user interface 370 can include x-axis slider 374, y-axis slider 376 and z-axis slider 378. Coordinate system icon 272 can provide an indication of the orientation of sensor module axis 264 of FIG. 9A. A user can utilize various inputs to adjust x-axis values 380, y-axis values 382 and z-axis values 384. For example, an input device such as a keyboard can be used to type values for x-axis values 380, y-axis values 382 and z-axis values 384; x-axis slider 374, y-axis slider 376 and z-axis slider 378 can be manipulated to increase or decrease x-axis values 380, y-axis values 382 and z-axis values 384; and coordinate system icon 372 can be manipulated, e.g., rotated, to adjust x-axis values 380, y-axis values 382 and z-axis values 384. As x-axis values 380, y-axis values 382 and z-axis values 384 are adjusted, the position of sensor module axis 264 relative to tibial axis 308 (FIG. 9A) can be adjusted on heads-up display screen 362 or another video display output. Thereafter, a controller for robotic system 115 can adjust the initial output of sensor module 240 after being implanted into the patient to align with tibial axis 308. A correction factor can be applied so that, for example, the x-axis values 380 will align with tibial axis 308 and y-axis values 382 and z-axis values 384 are orthogonal to the y-axis values, as discussed herein, so that the output of sensor module 240 is consistent with the anatomic frame of reference of the patient, e.g., the kinematic frame of reference defined by tibia 304. In other words, the coordinate system origin of sensor module 240 can be aligned with the coordinate system origin for the anatomy of the patient. Additionally, the x, y and z values for sensor module axis 264 can be adjusted electronically by tracking system 165 by tracking hand gestures using a motion-tracking system. For example, a user can pinch a virtual representation of sensor module axis 264 displayed by goggles 360 (FIG. 9A) and then make wrist or hand movements to rotate the representation of sensor module axis 264 to align with tibial axis 308. In examples, virtual “buttons” can be depressed via prescribed gestures to grab, rotate and release sensor module axis 264. In other examples, a handpiece can be held having buttons that electronically provide instructions to tracking system 165 for when a user grasps and releases sensor module axis 264.

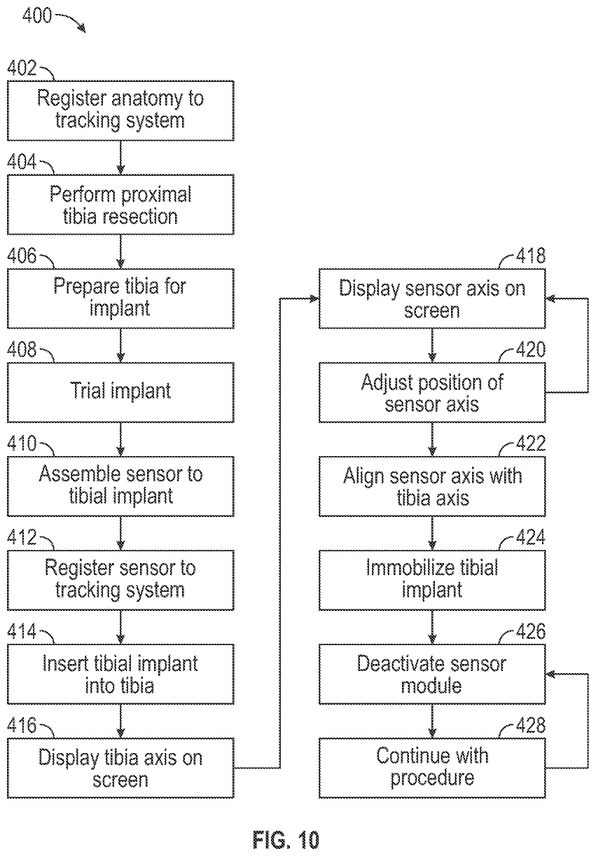

FIG. 10 is a block diagram of method 400 including various operations for exemplary methods of aligning sensor module 240 of tibial component 270 with tibia 304. Method 400 is described with reference to a tibia bone and a tibial implant for a total knee arthroplasty. However, method 400 can be adapted for implanting, aligning and calibrating sensor modules of other implants with other anatomy. Method 400 is described with reference to operation 402-operation 428. However, some of operation 402-operation 428 can be omitted and operation 402-operation 428 can be performed in other sequences.

At operation 402, anatomy of a patient can be registered to a tracking system for a surgical system, such as a robotic surgical system. For example, the anatomy of patient 110 (FIG. 1) can be registered to the three-dimensional space of surgical area 105. Tracking element 348 (FIG. 8) and tracking element 350 (FIG. 8) can be attached to the anatomy of patient 110, such as femur 302 (FIG. 8) and tibia 304 (FIG. 8). Thus, as described herein, the location and orientation of femur 302 and tibia 304 can be determined relative to a coordinate system of surgical area 105.

At operations 404 and 406, a bone can be prepared for receiving an implant having a sensor module. For example, at operation 404, the proximal end of tibia 304 can be resected to form proximal resection plane 312. As shown in FIG. 6, robotic arm 120 can be used to move resection guide instrument 200 proximate the proximal end of tibia 304 so that cutting device 280 can be engaged with guide surface 214 to form proximal resection plane 312 in a specific location and orientation on tibia 304. For example, at operation 406, proximal resection plane 312 can be prepared to receive tibial extension 276 and sensor module 240. As shown in FIG. 7, tibial drill guide 320 can be attached to tibia 304 so that a drill or reamer can be inserted into guide aperture 326 to form a bone channel or bone socket 279 (FIG. 4) to provide space for tibial extension 276 and sensor module 240 in a specific location and orientation on tibia 304. As such, proximal resection plane 312 and the bone channel or bone socket 279 can be formed within the three-dimensional space of surgical area 105 in such a manner that the orientation of tibial component 270 will be known to surgical system 100.

At operation 408, trial implants can be engaged with the prepared anatomy of the patient to determine, for example, the size and shape of the final construct. For example, a trial implant having the shape of tibial plate 272, keel 274 and tibial extension 276 can be engaged with tibia 304 at proximal resection plane 312 and bone socket 279. After trialing, a surgeon can determine the desired size of the implants for implanting into the patient to perform the prosthetic functionality.

At operation 410, sensor module 240 can be assembled with tibial component 270. For example, sensor module 240 can be connected to tibial extension 276 via suitable methods, such as force fit, snap fit and threaded engagement. Sensor module 240 can be rotated so that hash mark 266 (FIG. 4) aligns with hash mark 278 (FIG. 4).

At operation 412, sensor module 240 can be registered with surgical system 100. For example, sensor module 240 can be activated to communicate with base station 230 (FIG. 2). Orientation output of various sensors, e.g., accelerometers, gyroscopes and multi-axis sensors, of sensor module 240 can be integrated with the three-dimensional space of surgical area 105. That is three-dimensional x, y, z output of the orientation sensors can be superposed to the x, y, z coordinate system of surgical system 115, which can then be extrapolated and applied to the axis of tibia 304.

At operation 414, tibial component 270 can be implanted into tibia 304 as prepared in operation 404 and operation 406. Sensor module 240 and tibial extension 276 can be inserted into bone socket 279 and tibial plate 272 can be engaged with proximal resection plane 312. Bone cement or other material can be used to facilitate coupling of tibial component 270 to the anatomy.

At operation 416, tibial axis 308 can be displayed along tibia 304 for observation by a surgeon. Tibial axis 308 can be generated by the use of tracking element 348 and tracking element 350 (FIG. 8). In examples, tibial axis 308 can be displayed on one or both of human interface devices 145 (FIG. 1). In examples, tibial axis 308 can be projected onto tibia 304 via projector 344 (FIG. 8). In examples, tibial axis 308 can be displayed against an image of tibia 304 on heads-up display screen 362.

At operation 418, sensor module axis 264 can be displayed along sensor module 240 for observation by a surgeon. Sensor module axis 264 can be generated by the use of output of sensor module 240 being communicated to surgical system 115 via base station 230 (FIG. 2). In example, sensor module axis 264 can be displayed on one or both of human interface devices 145 (FIG. 1). In examples, sensor module axis 264 can be projected onto tibia 304 via projector 344 (FIG. 8). In examples, sensor module axis 264 can be displayed against an image of tibia 304 on heads-up display screen 362.

At operation 420, the position of sensor module axis 264 can be adjusted. Sensor module axis 264 can be adjusted by A) physically moving sensor module 240 either a) directly or b) via movement of tibial component 270 and B) digitally moving stem axis 314. As discussed with reference to FIG. 9B, sensor module axis 264 can be digitally adjusted by manipulating stem axis 314; coordinate system icon 372; x-axis values 380, y-axis values 382 and z-axis values 384; and x-axis slider 374, y-axis slider 376 and z-axis slider 378 on user interface 370 or by using motion-tracking of a surgeon in conjunction with a virtual representation of sensor module axis 264.

At operation 422, sensor module axis 264 can be adjusted to align with tibial axis 308. For example, the x-axis values 380 (FIG. 9B) can be aligned with tibial axis 308 and y-axis values 382 and z-axis values 384 are orthogonal to the y-axis values, as discussed herein, so that the output of sensor module 240 is consistent with the anatomic frame of reference of the patient, e.g., the kinematic frame of reference defined by tibia 304. For example, intended forward or anterior direction of sensor module 240, such as indicated by hash mark 266, can be aligned to the true forward or anterior direction of tibia 304 via a conversion of the output data of sensor module 240.

Operation 418 and operation 420 can be repeated until stem axis 314 is aligned with tibial axis 308. Once sensor module axis 264 is adjusted to the desired location. A correction factor can be applied to the output of sensor module 264 to shift the output as predisposed based on the implanted relationship to tibia 304 to align with tibial axis 308. The correction factor can be stored in memory of sensor module 264 so that future output of sensor module 240 communicated to base station 230 or another base station at the home of a patient can maintain the corrected or registered output.

At operation 424, tibial component 270 can be immobilized. For example, bone cement, which can be dispensed at operation 414, can be set so that tibial component 270 does not move and output of sensor module 240 will remain as registered.

At operation 426, sensor module 240 can be deactivated to preserve battery life. For example, an input into surgical system 115 can be communicated via base station 230 to sensor module 240. Additionally, other computing systems or handheld devices operating software for the control of sensor module 240 can be used to communicate with sensor module 240 via base station 230.

At operation 428, the surgical procedure can be continued and completed, leaving tibial component 270 along with sensor module 240 inside the patient. Incisions or access points within the anatomy can be closed to dispose tibial component 270 and sensor module 240 within the anatomy.

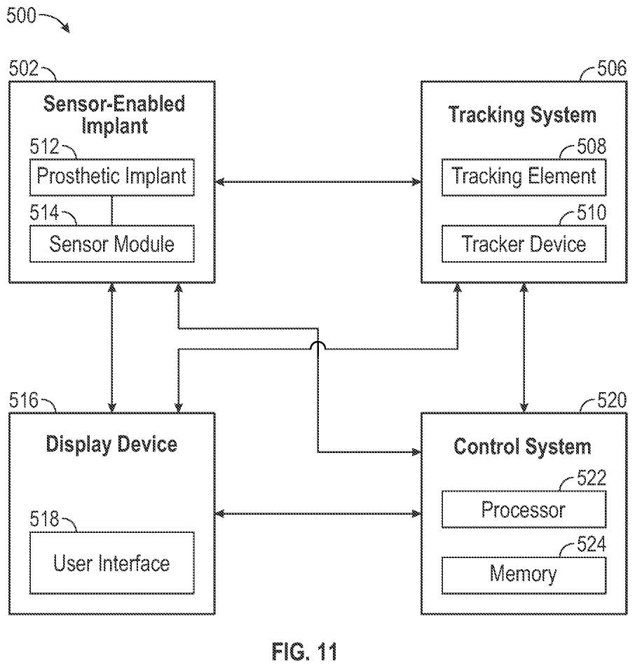

FIG. 11 illustrates system 500 for performing the methods, operations and techniques described herein, in accordance with some embodiments. System 500 is an example of a system that can incorporate surgical system 100 of FIG. 1. System 500 can include sensor-enabled implant 502, which can interact with tracking system 506. Sensor-enabled implant 502 can comprise prosthetic implant 512 (e.g., tibial component 270) and sensor module 514 (e.g., sensor module 240). In other examples, sensor-enabled implant 502 can be used without tracking system 506. Tracking system 506 can include tracking element 508 (e.g., tracking element 170) and tracker device 510 (e.g., a camera of tracking system 165). System 500 can include display device 516 (e.g., human interface device 145, a computer monitor or video display screen), which can be used with user interface 518 (e.g., a touchscreen, mouse or keyboard). System 500 can include control system 520 (e.g., a robotic controller or computing system 140 of FIG. 1), including processor 522 and memory 524. In an example, display device 516 can be coupled to one or more of sensor-enabled implant 502, tracking system 506, and control system 520. As such, data generated by sensor-enabled implant 502 can be shared with control system 520, tracking system 506 and an operator of system 500 via display device 516. In examples, sensor-enabled implant 502 can communicate with control system 520 via an external device, such as base station 230. In examples, sensor-enabled implant 502 can be operated without input from tracking system 506, after a registration process, such that sensor-enabled implant 502 can be positioned and tracked by movement of robotic arm 120 within the native coordinate system of robotic arm 120. Display device 516 can be used to visualize an axis of a bone determined by tracking system 506 and an axis of sensor module 514 determined by sensor module 514 and display both axes in a common frame of reference so that an operator of system 500 can ensure alignment of the axis either by physically moving prosthetic implant 512 and sensor module 514 to achieve alignment or by recalibrating the output of sensor module 514 to align the sensor axis with the anatomic axis. User interface 518 can be used to manipulate the orientation of the axis of sensor module 514. Control system 520 can be used to apply a correction factor to the output of sensor module 514 such as by programming or uploading the correction factor to sensor module 514.

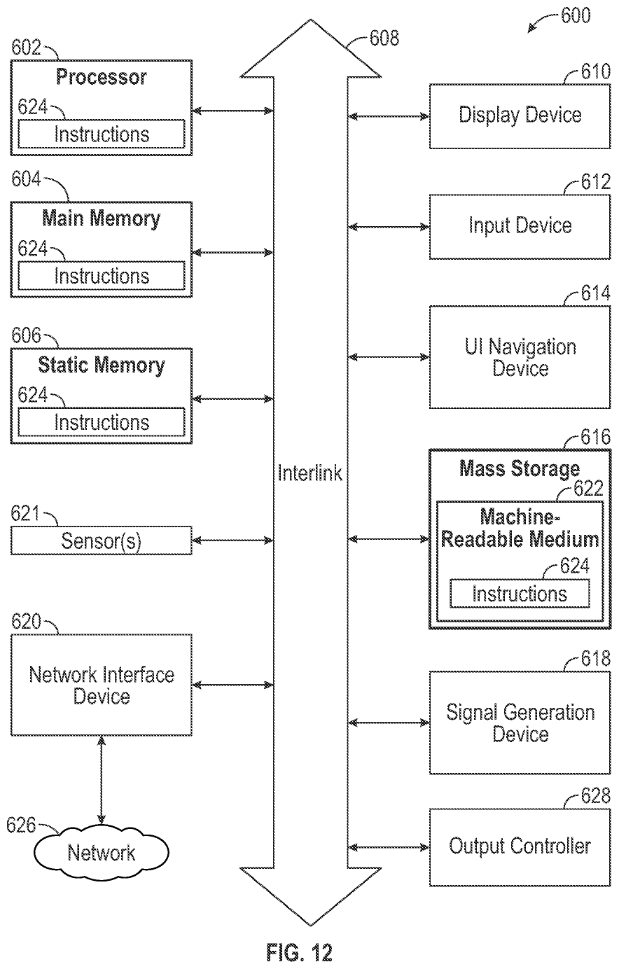

FIG. 12 illustrates a block diagram of example machine 600 upon which any one or more of the techniques discussed herein may be performed in accordance with some embodiments. For example, machine 600 can comprise computing system 140 of FIG. 1. Machine 600 can comprise an example of a controller for robotic system 115 and a sensor-enabled implant, such as the combination of tibial component 270 and sensor module 240, tracking element 348 and tracking element 350 and base station 230. As such instructions 624 can be executed by processor 602 to generate and correlate position and orientation information to determine the position and orientation of femur 302 and tibia 304 relative to robotic arm 120 and the position and orientation of sensor module 240 relative to robotic arm 120. Position and geometric information of sensor module 240 can be determined via implantation of tibial component 270 into tibia 304 in a known relationship according to a surgical plan. Geometric information, such as shapes, geometries and dimensions, for instruments connected to surgical arm and prosthetic implants can be stored in main memory 604 and accessed by processor 602. Furthermore, output of sensor module 240 can be provided to surgical system 100, e.g., at computing system 140 via connection to base station 230. Processor 602 can also receive input (such as at alphanumeric input device 612) relating to the position of tibia 304 relative to robotic arm 120 via tracking element 348 and tracking element 350, which can be stored in main memory 604. Processor 602 can further relate position information of sensor module 240 to the position information of robotic arm 120 by registering the spatial information output of sensor module 240 to the three-dimensional coordinate system of surgical system 100. Output of sensor module 240 can be additionally registered to the anatomy and specifically the bone into which tibial component 270 is implanted. Thus, as femur 302 and tibia 304 move through a range of motion, machine 600 can continuously track and update the location of sensor module 240 relative to the three-dimensional space of surgical system 100. Machine 600 can additionally display the positions of femur 302, tibia 304, sensor module 240 and axes thereof on display unit 610 (e.g., human interface devices 145), as well as the location of features included thereon, such as cutting guide features. User interface navigation device 614 can be used to adjust the output of sensor module 240 to that sensor module axis 240 aligns with tibial axis 308.

In alternative embodiments, machine 600 may operate as a standalone device or may be connected (e.g., networked) to other machines. In a networked deployment, machine 600 may operate in the capacity of a server machine, a client machine, or both in server-client network environments. In an example, machine 600 may act as a peer machine in peer-to-peer (P2P) (or other distributed) network environment. Machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include processor 602 (e.g., a central processing unit (CPU), a graphics processing unit (GPU), a hardware processor core, or any combination thereof), main memory 604 and static memory 606, some or all of which may communicate with each other via interlink 608 (e.g., a bus). Machine 600 may further include display unit 610, alphanumeric input device 612 (e.g., a keyboard), and user interface navigation device 614 (e.g., a mouse). In an example, display unit 610, alphanumeric input device 612 and user interface navigation device 614 may be a touch screen display. Machine 600 may additionally include storage device 616 (e.g., a drive unit), signal generation device 618 (e.g., a speaker), network interface device 620, and one or more sensors 621, such as a global positioning system (GPS) sensor, compass, accelerometer, or other sensor. Machine 600 may include output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

Storage device 616 may include machine readable medium 622 on which is stored one or more sets of data structures or instructions 624 (e.g., software) embodying or utilized by any one or more of the techniques or functions described herein. Instructions 624 may also reside, completely or at least partially, within main memory 604, within static memory 606, or within processor 602 during execution thereof by machine 600. In an example, one or any combination of processor 602, main memory 604, static memory 606, or storage device 616 may constitute machine readable media.

While machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by machine 600 and that cause machine 600 to perform any one or more of the techniques of the present disclosure, or that is capable of storing, encoding or carrying data structures used by or associated with such instructions. Non-limiting machine readable medium examples may include solid-state memories, and optical and magnetic media.

Instructions 624 may further be transmitted or received over communications network 626 using a transmission medium via network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, network interface device 620 may include one or more physical jacks (e.g., Ethernet, coaxial, or phone jacks) or one or more antennas to connect to communications network 626. In an example, network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by machine 600, and includes digital or analog communications signals or other intangible medium to facilitate communication of such software.

The systems, devices and methods discussed in the present application can be useful in performing robotic-assisted surgical procedures that utilize robotic surgical arms that can be used to position devices relative to a patient to perform arthroplasty procedures, such as partial knee arthroplasties. In particular, the systems, devices and methods disclosed herein are useful in registering output of sensor-enabled implants to the three-dimensional space of robotic surgical systems to determine, verify, offset and calibrate output of the sensor-enabled implant to the anatomy of a patient so that the output of the sensor-enabled implant is reflective or indicative of real-world kinematic movements of the anatomy of the patient. The systems, devices and methods disclosed herein can reduce or eliminate errors from sensor output that is skewed from anatomy of a patient that can arise from improperly assembled sensor modules and prosthetic devices, improperly implanted prosthetic devices or slight deviations from surgical plans due to operator variance or anatomic imperfections or anomalies. As such, output of the sensor-enabled implant can be utilized to provide accurate feedback to a patient and a surgeon regarding the operation or movements of a joint in which a sensor-enabled implant is implanted to verify or determine the effectiveness of the implant and to prescribe actions for the patient to overcome or avoid pain, discomfort and the like.

### Examples

Example 1 is a method for registering output of a sensor-enabled implant with a bone axis during a robotically-assisted arthroplasty procedure, the method comprising: registering anatomy of a patient to a surgical tracking system; determining a bone axis of a bone of the anatomy using the surgical tracking system; preparing the bone to receive a prosthetic implant including an orientation sensor; inserting the prosthetic implant into the bone; obtaining orientation output from the orientation sensor; and shifting the orientation output from the orientation sensor to align with the bone axis.

In Example 2, the subject matter of Example 1 optionally includes wherein shifting the orientation output from the orientation sensor to align with the bone axis comprises: aligning one axis of a three-dimensional coordinate system of the orientation sensor to align with the bone axis.

In Example 3, the subject matter of any one or more of Examples 1-2 optionally include wherein shifting the orientation output from the orientation sensor comprises: manually adjusting a position of the prosthetic implant in the bone.

In Example 4, the subject matter of any one or more of Examples 2-3 optionally include wherein shifting the orientation output from the orientation sensor comprises: manually adjusting a position of the orientation sensor relative to the prosthetic implant.

In Example 5, the subject matter of any one or more of Examples 1-4 optionally include wherein shifting the orientation output from the orientation sensor comprises: digitally adjusting the orientation output to align with the bone axis.

In Example 6, the subject matter of Example 5 optionally includes wherein digitally adjusting the orientation output to align with the bone axis: applying a mathematical correction factor to the orientation output.

In Example 7, the subject matter of Example 6 optionally includes wherein applying a mathematical correction factor to the orientation output comprises: automatically applying the mathematical correction factor with a controller of the surgical tracking system.

In Example 8, the subject matter of Example 7 optionally includes wherein applying a mathematical correction factor to the orientation output comprises: displaying a digital representation of the bone axis on an output device of the surgical tracking system; displaying a digital representation of a sensor axis of the orientation sensor on the output device of the surgical tracking system; and manually shifting orientation of the digital representation of the sensor axis to align with the digital representation of the bone axis using an input device of the surgical tracking system.

In Example 9, the subject matter of Example 8 optionally includes wherein manually shifting orientation of the digital representation of the sensor axis to align with the digital representation of the bone axis using an input device of the surgical tracking system comprises: using a touchscreen to adjust a position of the digital representation of the sensor axis.

In Example 10, the subject matter of any one or more of Examples 8-9 optionally include wherein manually shifting orientation of the digital representation of the sensor axis to align with the digital representation of the bone axis using an input device of the surgical tracking system comprises: using a touchscreen to adjust numerical values associated with an X, Y and Z position of the digital representation of the sensor axis.

In Example 11, the subject matter of any one or more of Examples 8-10 optionally include wherein the output device comprises an augmented reality headset.

Example 12 is a system for registering output of a sensor-enabled implant with a bone axis during a robotically-assisted arthroplasty procedure, the system comprising: a surgical robot comprising an articulating arm configured to move within a coordinate system for the surgical robot; a tracking system configured determine locations of one or more trackers in the coordinate system; a sensor-enabled implant configured to be implanted into anatomy and output orientation data; and a controller for the surgical robot, the controller comprising: a communication device configured to receive data from and transmit data to the surgical robot, the tracking system and the sensor-enabled implant; a display device for outputting visual information from the surgical robot, the tracking system and the sensor-enabled implant; and a non-transitory storage medium having computer-readable instructions stored therein comprising: registering anatomy of a patient to a surgical tracking system; determining a bone axis of a bone of the anatomy using the surgical tracking system; obtaining orientation output from an orientation sensor of a sensor-enabled prosthetic implant implanted into bone; and shifting the orientation output from the orientation sensor to align with the bone axis.

In Example 13, the subject matter of Example 12 optionally includes wherein shifting the orientation output from the orientation sensor to align with the bone axis comprises: aligning one axis of a three-dimensional coordinate system of the orientation sensor to align with the bone axis.

In Example 14, the subject matter of any one or more of Examples 12-13 optionally include wherein shifting the orientation output from the orientation sensor comprises: digitally adjusting the orientation output to align with the bone axis.

In Example 15, the subject matter of Example 14 optionally includes wherein digitally adjusting the orientation output to align with the bone axis: applying a mathematical correction factor to the orientation output.

In Example 16, the subject matter of Example 15 optionally includes wherein applying a mathematical correction factor to the orientation output comprises: automatically applying the mathematical correction factor with a controller of the surgical tracking system.

In Example 17, the subject matter of Example 16 optionally includes wherein applying a mathematical correction factor to the orientation output comprises: displaying a digital representation of the bone axis on an output device of the surgical tracking system; displaying a digital representation of a sensor axis of the orientation sensor on the output device of the surgical tracking system; and receiving a manual shift in orientation of the digital representation of the sensor axis to align with the digital representation of the bone axis using an input device of the surgical tracking system.

In Example 18, the subject matter of Example 17 optionally includes wherein receiving a manual shift in orientation of the digital representation of the sensor axis to align with the digital representation of the bone axis using an input device of the surgical tracking system comprises: receiving an input from a touchscreen or a gesture-tracking system to adjust a position of the digital representation of the sensor axis.

In Example 19, the subject matter of any one or more of Examples 17-18 optionally include wherein receiving a manual shift in orientation of the digital representation of the sensor axis to align with the digital representation of the bone axis using an input device of the surgical tracking system comprises: receiving an input from a touchscreen or a gesture-tracking system to adjust numerical values associated with an X, Y and Z position of the digital representation of the sensor axis.

In Example 20, the subject matter of any one or more of Examples 12-19 optionally include wherein the display device comprises an augmented reality headset.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method for registering output of a sensor-enabled implant with a bone axis during a robotically-assisted arthroplasty procedure, the method comprising:
registering anatomy of a patient to a surgical tracking system;
determining a bone axis of a bone of the anatomy using the surgical tracking system;
preparing the bone to receive a prosthetic implant including an orientation sensor;
inserting the prosthetic implant into the bone;
obtaining orientation output from the orientation sensor; and
shifting the orientation output from the orientation sensor to align with the bone axis.

2. The method of claim 1, wherein shifting the orientation output from the orientation sensor to align with the bone axis comprises:
aligning one axis of a three-dimensional coordinate system of the orientation sensor to align with the bone axis.

3. The method of claim 1, wherein shifting the orientation output from the orientation sensor comprises:
manually adjusting a position of the prosthetic implant in the bone.

4. The method of claim 2, wherein shifting the orientation output from the orientation sensor comprises:
manually adjusting a position of the orientation sensor relative to the prosthetic implant.

5. The method of claim 1, wherein shifting the orientation output from the orientation sensor comprises:
digitally adjusting the orientation output to align with the bone axis.

6. The method of claim 5, wherein digitally adjusting the orientation output to align with the bone axis:
applying a mathematical correction factor to the orientation output.

7. The method of claim 6, wherein applying a mathematical correction factor to the orientation output comprises:
automatically applying the mathematical correction factor with a controller of the surgical tracking system.

8. The method of claim 7, wherein applying a mathematical correction factor to the orientation output comprises:
displaying a digital representation of the bone axis on an output device of the surgical tracking system;
displaying a digital representation of a sensor axis of the orientation sensor on the output device of the surgical tracking system; and
manually shifting orientation of the digital representation of the sensor axis to align with the digital representation of the bone axis using an input device of the surgical tracking system.

9. The method of claim 8, wherein manually shifting orientation of the digital representation of the sensor axis to align with the digital representation of the bone axis using an input device of the surgical tracking system comprises:
using a touchscreen to adjust a position of the digital representation of the sensor axis.

10. The method of claim 8, wherein manually shifting orientation of the digital representation of the sensor axis to align with the digital representation of the bone axis using an input device of the surgical tracking system comprises:
using a touchscreen to adjust numerical values associated with an X, Y and Z position of the digital representation of the sensor axis.

11. The method of claim 8, wherein the output device comprises an augmented reality headset.

12. A system for registering output of a sensor-enabled implant with a bone axis during a robotically-assisted arthroplasty procedure, the system comprising:
a surgical robot comprising an articulating arm configured to move within a coordinate system for the surgical robot;
a tracking system configured determine locations of one or more trackers in the coordinate system;
a sensor-enabled implant configured to be implanted into anatomy and output orientation data; and
a controller for the surgical robot, the controller comprising:
a communication device configured to receive data from and transmit data to the surgical robot, the tracking system and the sensor-enabled implant;
a display device for outputting visual information from the surgical robot, the tracking system and the sensor-enabled implant; and
a non-transitory storage medium having computer-readable instructions stored therein comprising:
registering anatomy of a patient to a surgical tracking system;
determining a bone axis of a bone of the anatomy using the surgical tracking system;
obtaining orientation output from an orientation sensor of a sensor-enabled prosthetic implant implanted into bone; and
shifting the orientation output from the orientation sensor to align with the bone axis.

13. The system of claim 12, wherein shifting the orientation output from the orientation sensor to align with the bone axis comprises:
aligning one axis of a three-dimensional coordinate system of the orientation sensor to align with the bone axis.

14. The system of claim 12, wherein shifting the orientation output from the orientation sensor comprises:
digitally adjusting the orientation output to align with the bone axis.

15. The system of claim 14, wherein digitally adjusting the orientation output to align with the bone axis:
applying a mathematical correction factor to the orientation output.

16. The system of claim 15, wherein applying a mathematical correction factor to the orientation output comprises:
automatically applying the mathematical correction factor with a controller of the surgical tracking system.

17. The system of claim 16, wherein applying a mathematical correction factor to the orientation output comprises:
displaying a digital representation of the bone axis on an output device of the surgical tracking system;
displaying a digital representation of a sensor axis of the orientation sensor on the output device of the surgical tracking system; and
receiving a manual shift in orientation of the digital representation of the sensor axis to align with the digital representation of the bone axis using an input device of the surgical tracking system.

18. The system of claim 17, wherein receiving a manual shift in orientation of the digital representation of the sensor axis to align with the digital representation of the bone axis using an input device of the surgical tracking system comprises:
receiving an input from a touchscreen or a gesture-tracking system to adjust a position of the digital representation of the sensor axis.

19. The system of claim 17, wherein receiving a manual shift in orientation of the digital representation of the sensor axis to align with the digital representation of the bone axis using an input device of the surgical tracking system comprises:
receiving an input from a touchscreen or a gesture-tracking system to adjust numerical values associated with an X, Y and Z position of the digital representation of the sensor axis.

20. The system of claim 12, wherein the display device comprises an augmented reality headset.
